(19) United States
(12) Patent Application Publication
(10) Pub. No.: US 2025/0069718 A1
(43) Pub. Date: Feb. 27, 2025

(54) INTELLIGENT WELLNESS MANAGEMENT PLATFORM

(71) Applicant: Main Grade Assets, LLC, Orlando, FL (US)

(72) Inventors: James Beach-Drummond, Orlando, FL (US); Anthony Rivera, Orlando, FL (US); Carmin Vaccaro, Orlando, FL (US); Robert Hoppenfeld, Orlando, FL (US)

(21) Appl. No.: 18/810,380

(22) Filed: Aug. 20, 2024

Related U.S. Application Data
(60) Provisional application No. 63/520,701, filed on Aug. 21, 2023.

(51) Int. Cl. G16H 20/00

(52) U.S. Cl. CPC G16H 20/00

(57) ABSTRACT

A system and method are disclosed for an intelligent wellness management platform that integrates multiple wellness dimensions into a unified, adaptive system. Using advanced AI, the platform creates personalized wellness journeys that adapt in real time to users' changing goals and external influences, ensuring relevance and effectiveness. A virtual advisor guides users through goal setting, data entry, and provides continuous support for a seamless experience. The platform also identifies user connections through “orbital convergence,” enabling meaningful interactions and shared experiences to enhance wellness outcomes. With real-time data processing, event-driven architecture, and user feedback, the system continuously updates recommendations, allowing users to manage their wellness journey with personalized guidance. The platform empowers users to customize their wellness paths, set goals and timeframes, and participate in community-driven activities, all while maintaining autonomy and flexibility.

### RELATED APPLICATION

The present application claims the benefit of U.S. Provisional Patent Application No. 63/520,701, filed on Aug. 21, 2023, which is incorporated herein by reference in its entirety.

### TECHNICAL FIELD

The present disclosure relates to holistic wellness and well-being enhancement, and, more particularly, within the context of digital health platforms powered by artificial intelligence.

### BACKGROUND

In today's increasingly connected world, there is a growing recognition of the importance of a holistic approach to wellness. The dimensions of mental, physical, spiritual, financial, educational, and social well-being are deeply interwoven, each impacting the other.

The challenge, however, lies in effectively managing these dimensions in a way that recognizes their interconnectedness and adapts to the unique needs of each individual. Despite the advances in digital health technologies, many existing solutions remain compartmentalized, focusing on isolated aspects of wellness rather than offering a truly integrated approach. Users often find themselves juggling multiple tools and platforms, none of which fully capture the complexity of their wellness journey.

Moreover, these solutions often lack the ability to dynamically adapt to the user's evolving goals and external influences, leading to a fragmented and sometimes ineffective wellness experience. A comprehensive and adaptable solution is needed. One that not only integrates all dimensions of wellness but also responds in real-time to the user's progress and changing circumstances. Such a solution would empower users to take control of their wellness journey, providing personalized guidance and support that evolves with them.

### SUMMARY

System and methods that integrate multiple dimensions of wellness-mental, physical, spiritual, financial, educational, and social into a unified and dynamic software platform are provided. The software platform leverages advanced artificial intelligence (AI) techniques, including natural language processing and real-time data analysis, to provide personalized wellness journeys that adapt to individual user needs and external influences.

The software platform also encompasses the identification of synergies among users through a concept known as “orbital convergence,” which facilitates meaningful interactions and connections, thereby enhancing overall user outcomes in their wellness pursuits.

The software platform enhances holistic wellness across multiple dimensions based on advanced AI to monitor and analyze user interactions, and on offering personalized wellness journeys that are continuously adjusted to reflect the user's progress and external factors. By recognizing synergies among users, the platform fosters meaningful connections and shared experiences, further enriching the wellness journey. By providing a truly integrated and responsive solution, users are enabled to achieve a balanced and fulfilling life, addressing the complexities of modern wellness in a way that is both comprehensive and personalized.

One aspect is directed to a system that introduces an innovative software platform designed to enhance and manage holistic wellness across multiple dimensions of well-being. This platform leverages the power of AI to provide users with a personalized, dynamic, and comprehensive wellness journey that may adapt in real-time to their evolving needs and external influences.

In some embodiments, the system analyzes vast amounts of user data, including personal goals, preferences, and interactions. The AI engine may process this data to generate individualized wellness paths, or “wellness journeys,” that guide users toward achieving their specific objectives. These wellness journeys are not necessarily static. They may be continuously monitored and adjusted by the AI to ensure alignment with the user's progress and any changes in their circumstances.

The system may also include a feature referred to as “orbital convergence.” For example, in some embodiments, this concept involves the identification of synergies and intersections between the wellness journeys of different users. When the AI detects that users have overlapping goals or complementary needs, the system may facilitate connections between them, enabling shared experiences, peer support, and collaboration. This feature not only enriches the user experience but also enhances the overall effectiveness of the wellness journey by introducing community-driven elements.

Additionally, in some embodiments, the platform may be highly responsive to external factors, such as new research, emerging wellness trends, and global events. The AI may incorporate these influences into its analysis, updating recommendations and wellness paths to ensure they remain relevant and beneficial in the broader wellness landscape.

For case of use, the user interface may be designed for simplicity and accessibility, featuring an AI-powered chatbot that may guide users through the onboarding process, help them set goals, and provide ongoing support. The interface may support multiple languages and be optimized for both mobile and desktop devices, ensuring that users can engage with their wellness journey anytime, anywhere.

In view of the above, one aspect is directed to a wellness management platform for facilitating user interactions based on shared wellness goals. The wellness management platform includes a computing device and a database coupled to the computing device.

The database may be configured to store wellness data on a plurality of users enrolled in the wellness management platform. Each user may be pursuing a personalized wellness journey generated by the wellness management platform to achieve at least one wellness goal, with the at least one wellness goal for each user having a progress status associated therewith that is being tracked and updated by the wellness management platform based on accomplishments of each user.

The computing device may be configured to perform the following steps. A new user is enrolled in the wellness management platform, wherein the new user selects at least one wellness goal from a plurality of wellness dimensions. A personalized wellness journey may be generated for the new user, with the personalized wellness journey providing recommendations to achieve the selected at least one wellness goal.

Progress of the new user on their personalized wellness journey is tracked based on accomplishments of the new user. The tracked progress of the new user on their personalized wellness journey is compared to the wellness data stored in the database to identify a convergence with at least one of the users participating in the wellness management platform. The identified convergence may correspond to overlapping or complementary wellness goals between the new user and the at least one user associated with the convergence.

A suggestion for the new user is generated based on the identified convergence, wherein the suggestion includes at least one recommendation for joint activities, shared resources or communications with the at least one user associated with the identified convergence. The generated suggestion is communicated to the new user over a communications channel, and user options are provided via the generated suggestion to allow the new user to accept, modify, or decline the generated suggestion.

The computing device may include a virtual advisor to assist the new user in selecting the at least one wellness goal, to define wellness objectives, and to input relevant data for generation of the personalized wellness journey. The virtual advisor may be a chatbot that uses natural language processing to assist the new user.

The computing device includes an artificial intelligence (AI) module to generate the personalized wellness journey, track progress of the new user on their personalized wellness journey, identify the convergence, and generate the suggestion for the new user based on the identified convergence. The AI module may be further configured to make adjustments in the personalized wellness journey based on real-time feedback and progress indicators from the new user.

The plurality of wellness dimensions may include mental wellness, physical wellness, spiritual wellness, financial wellness, educational wellness and social wellness. The recommendation for the new user may further include activities for the new user to perform for each selected goal within one or more of the wellness dimensions. The computing device may be configured to allow the user to set specific timeframes for achieving each selected goal within each wellness dimension, and dynamically adjust the recommended activities based on the selected timeframes.

The computing device may be configured to generate notifications related to the personalized wellness journey of the new user, with the notifications comprising at least one of reminders, motivational messages, and progress updates.

The generated suggestion for the new user based on the identified convergence may further include recommendations for one or more wellness related activities or events.

Identifying the convergence may be based on at least one of user-defined wellness objectives, historical progress data, user preferences for interaction, and external wellness-related events or opportunities. The user preferences for interaction may include virtual meetings, shared wellness activities, mentoring or peer support relationships, collaborative learning sessions, and community event participation.

The computing device may be configured to prioritize the convergence based on a degree of complementarity between the at least one goal of the new user and the goals of the users in the database, with higher priority given to convergences that are likely to result in mutually beneficial outcomes.

The computing device may be configured to track effectiveness of interactions facilitated by the convergence, and to use this data to refine future recommendations and improve accuracy of convergence identification.

An interface may provide a user-controlled option to manually search for potential convergences based on specific criteria, such as location, wellness focus, or desired interaction type. The generated suggestion may be communicated over the communications channel to the new user via one or more of mobile app notifications, emails, and SMS messages.

Another aspect is directed to a method for facilitating user interactions based on shared wellness goals. The method includes storing in a database wellness data on a plurality of users enrolled in a wellness management platform. Each user may be pursuing a personalized wellness journey generated by the wellness management platform to achieve at least one wellness goal, with the at least one wellness goal for each user having a progress status associated therewith that is being tracked and updated by the wellness management platform based on accomplishments of each user.

The method further includes enrolling a new user in the wellness management platform, wherein the new user selects at least one wellness goal from a plurality of wellness dimensions. A personalized wellness journey for the new user is generated, with the personalized wellness journey providing recommendations to achieve the selected at least one wellness goal.

The method further includes tracking progress of the new user on their personalized wellness journey based on accomplishments of the new user. The tracked progress of the new user on their personalized wellness journey may be compared to the wellness data stored in the database to identify a convergence with at least one of the users participating in the wellness management platform, wherein the identified convergence corresponds to overlapping or complementary wellness goals between the new user and the at least one user associated with the convergence.

A suggestion is generated for the new user based on the identified convergence, wherein the suggestion includes at least one recommendation for joint activities, shared resources or communications with the at least one user associated with the identified convergence. The generated suggestion is communicated to the new user over a communications channel, with user options being provided via the generated suggestion to allow the new user to accept, modify, or decline the generated suggestion.

Yet another aspect is directed to a non-transitory computer readable medium having computer-executable instructions for causing a wellness management platform to perform the above method steps.

### BRIEF DESCRIPTION OF THE DRAWINGS

FIG. 1.0 illustrates an overall graphic depiction of the Wellness Management System, showcasing the integration of a wellness management platform with users' personal digital devices.

FIG. 1.1 provides a detailed block diagram of the Wellness Management Platform, highlighting its core components, including the data storage, process and procedure engine, and various interfaces.

FIG. 1.2 depicts an embodiment of the user interface and initial onboarding process of the wellness platform.

FIG. 1.3 shows the process of real-time interactions using Bluetooth-connected devices.

FIG. 1.4 depicts real-time interactions using locally installed applications on the user's device.

FIG. 1.5 illustrates real-time interactions that use web-based connections to link the platform with a wide range of external data sources, such as geolocation data, community events, online wellness content, and social media.

FIG. 2.0 illustrates the workflow of a virtual advisor system within the wellness platform, where users define their wellness objectives, input relevant data, and the AI engine processes this information to create and adapt personalized wellness journeys.

FIG. 3.0 presents the concept of a “personal orbit” within the wellness journey, showing how the AI-enhanced virtual advisor engages with users and continuously refines their wellness trajectory, in accordance with an embodiment of the present disclosure.

FIG. 3.1 provides a detailed view of the “personal orbit,” illustrating how users' wellness journeys are structured around specific goals, and how the AI adjusts the user's orbit in response to real-time data, in accordance with an embodiment of the present disclosure.

FIGS. 4.0-4.4 illustrate the concept of “orbital convergence” in accordance with an embodiment of the present disclosure.

FIG. 5.0 depicts the continuous evaluation process, where the AI monitors and adjusts recommendations based on user progress and interactions, in accordance with an embodiment of the present disclosure.

FIG. 5.1 expands on continuous evaluation by showing how the platform integrates real-time external factors through AI algorithms and event-driven architecture to update and personalize the user's wellness journey.

FIG. 6.0 illustrates the holistic approach to wellness, showing the interconnectedness of various wellness dimensions and their integration into a unified experience, in accordance with an embodiment of the present disclosure.

### DETAILED DESCRIPTION

One or more specific embodiments of the present disclosure are described below. In an effort to provide a concise description of these embodiments, certain features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another.

Moreover, it should be appreciated that such a development effort might be complex and time-consuming, but would nevertheless be a routine undertaking of design, fabrication, and manufacture for those of ordinary skill having the benefit of this disclosure.

While only certain features of the disclosure have been illustrated and described herein, many modifications and changes will occur to those skilled in the art. For example, while the embodiments described herein include a specific logic organization for wellness management system services, substantially similar benefits provided by the present invention may be obtained by trivial structural alterations such as changes in name or in non-substantial changes to the mode of operation of the data structures. It is, thus, to be understood that the appended claims are intended to cover all such modifications and changes as fall within the true spirit of the invention.

In addition to the specific features and embodiments described herein, the present invention broadly encompasses systems and methods that integrate multiple dimensions of wellness, including but not limited to mental, physical, spiritual, financial, and educational applications. The invention is adaptable for use across a wide range of fields, including wellness platforms for corporate employees, military veterans, students, elderly individuals, individuals with chronic illnesses, parents, preadults, athletes, teachers and educators, and individuals facing mental and physical health challenges, among others. By leveraging a dynamic and customizable framework, the invention provides a holistic approach to wellness that can be adapted to the unique needs and goals of diverse user populations. This adaptability ensures that the wellness management system can be applied in various contexts, supporting users through personalized journeys that reflect their individual circumstances and challenges. The system's capacity to incorporate real-time data and user feedback further enhances its applicability across different fields, allowing it to respond dynamically to changing needs and emerging trends within each specific user group. Accordingly, the invention is not limited to the specific embodiments described but extends to all applications and modifications that achieve similar benefits in the broader context of wellness management.

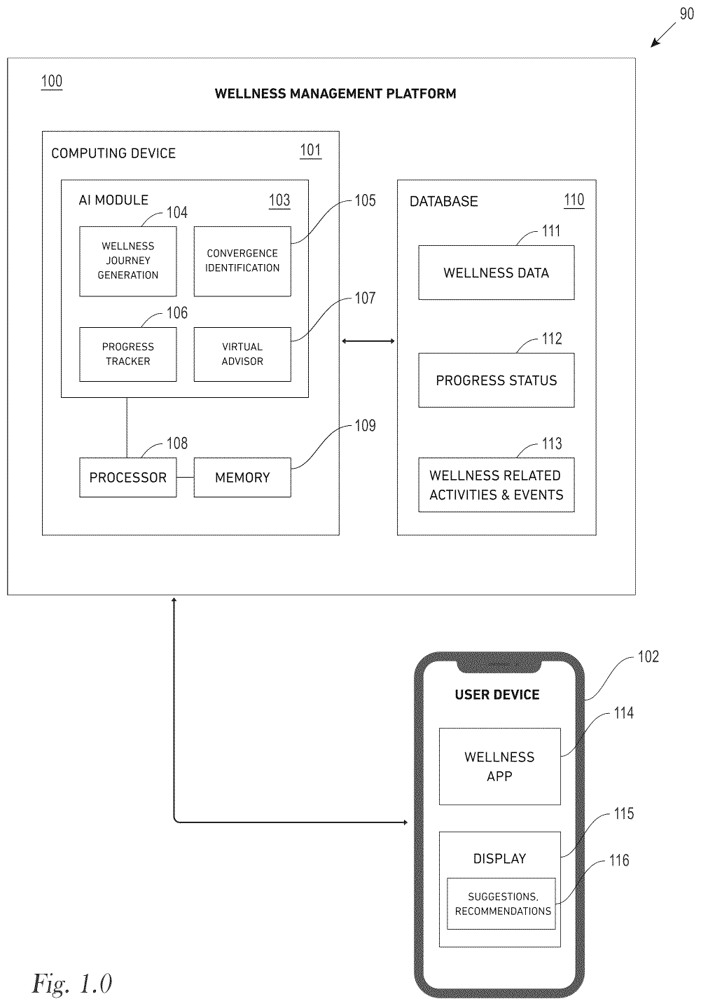

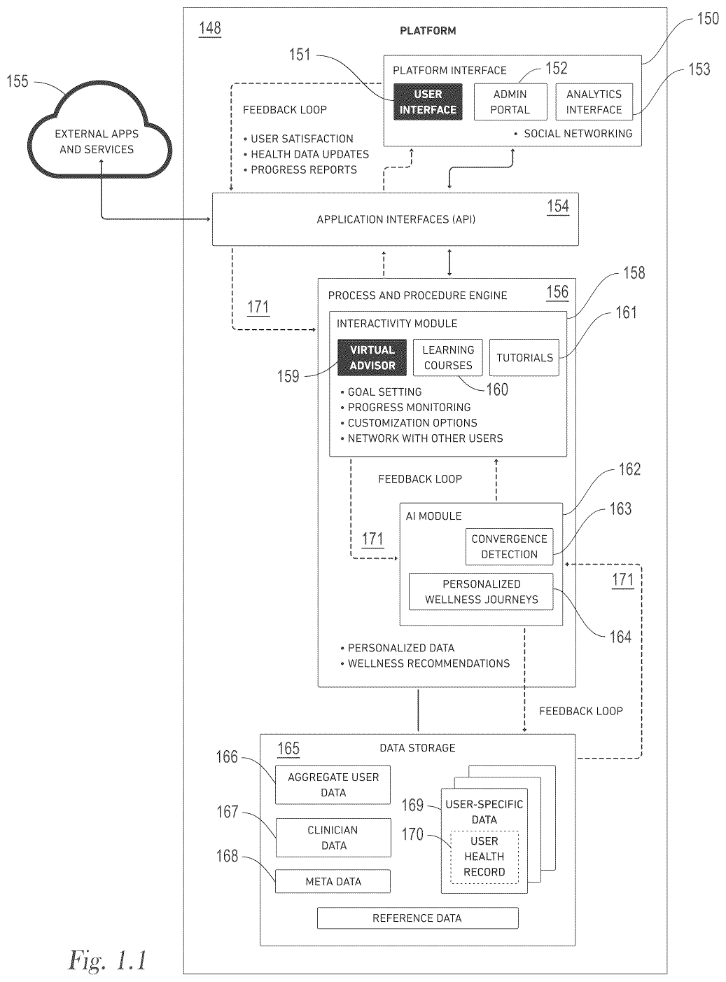

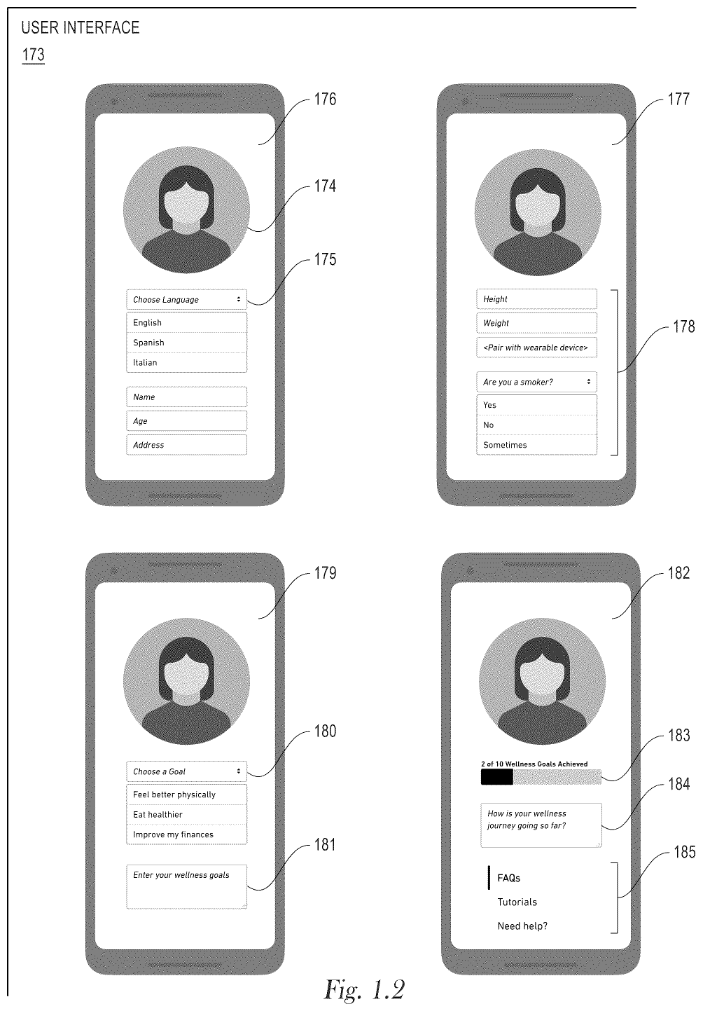

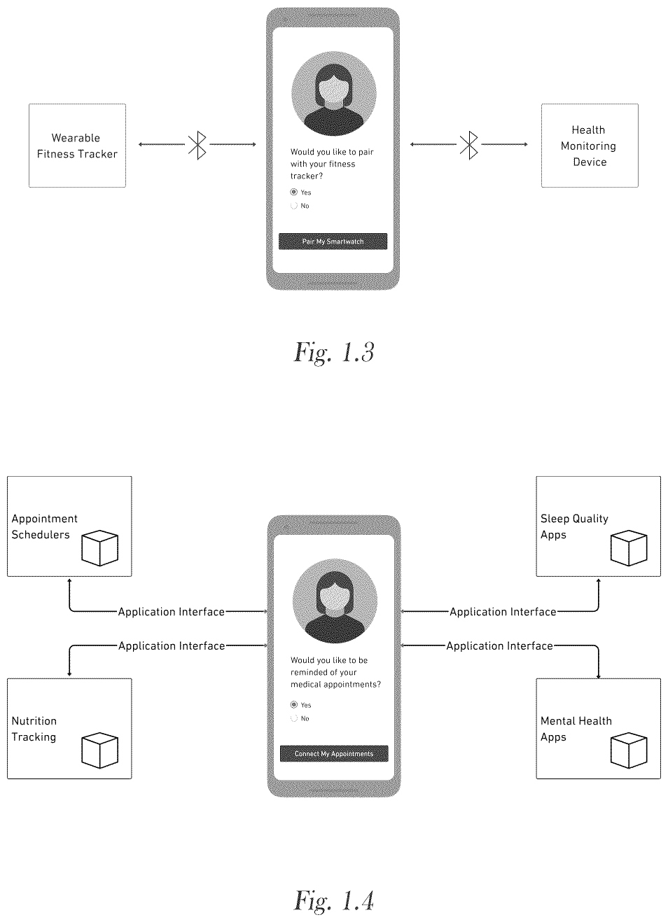

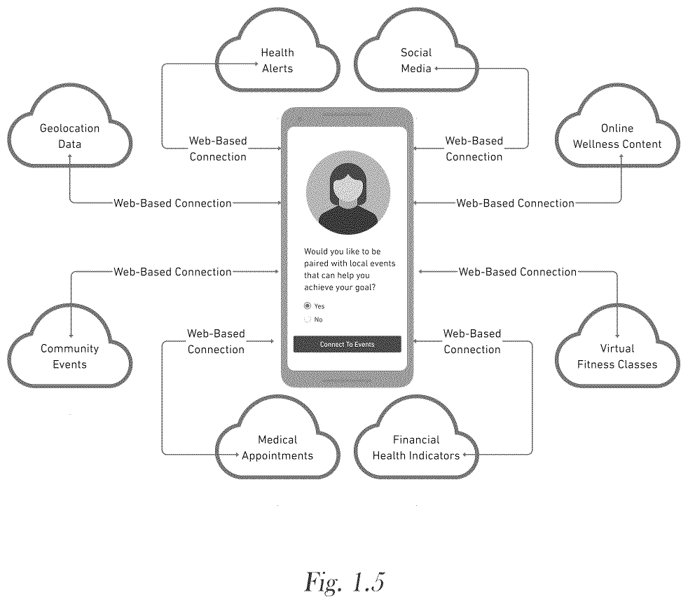

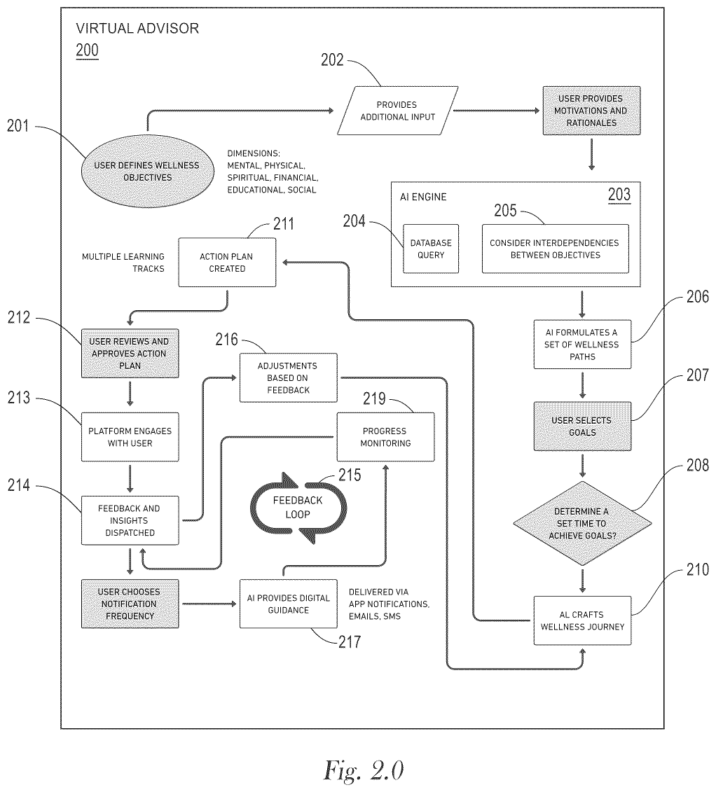

Referring now to FIG. 1.0, a wellness management system 90 includes a wellness management platform 100 interfacing with a user device 102. The wellness management platform may facilitate user interactions based on shared wellness goals or based on wellness related activities or events.

Even though only one user device 102 is shown, a plurality of different user devices may interface with the wellness management platform 100. The user device 102 may be a desktop computer, a laptop computer, a tablet computer, a mobile computing device, for example. As will be discussed in greater detail below, the wellness management platform 100 integrates various AI-driven modules and user devices to manage and optimize users' wellness journeys.

The computing device 101 houses the core AI module 103 responsible for generating personalized wellness journeys, identifying points of convergence between users, tracking user progress, and managing virtual interactions with the user through a virtual advisor. The illustrated AI module 103 includes a wellness journey generation module 104, a convergence identification module 105, a progress tracker 106 and a virtual advisor 107.

The wellness journey generation module 104 is tasked with creating individualized wellness paths based on the user's inputs and preferences. This module may utilize complex algorithms to continuously refine and adjust the wellness journeys based on real-time data and user feedback.

The convergence identification module 105 is responsible for detecting intersections or synergies between users' wellness journeys. This feature enables community-driven support and collaborative wellness activities among users. The progress tracker 106 is to monitor the user's progress against their set wellness goals. It provides ongoing updates and adjustments to the wellness journey, ensuring alignment with the user's evolving needs. Additionally, the virtual advisor 107 acts as an interactive interface that guides users through their wellness journey, offering advice, support, and recommendations based on Al analysis.

The processor 108 and memory 109 components are crucial to the computing device 101, enabling it to perform complex computations, store vast amounts of user and wellness-related data, and maintain seamless functionality across all modules. The computing device 101 is coupled to a database 110. The database 110 may be collocated with the computing device 101, as shown, or may be remote from the computing device 101.

The database 110 communicates with the computing device 101 to store and manage wellness data 111 and progress status 112. This database is responsible for housing a wide array of information, including user health metrics, activity logs, preferences, and wellness goals. The wellness data 111 stored in the database includes detailed records of users' health conditions, fitness levels, and other personal metrics that inform the AI module's decision-making processes.

The progress status 112 section of the database keeps track of each user's journey, recording their achievements and areas needing improvement, which can be referenced for future recommendations. The wellness-related activities and events 113 component of the database ensures that users are kept informed of relevant wellness opportunities, such as workshops, seminars, or fitness classes that align with their wellness goals.

By storing this information, the database enables the AI module to suggest personalized activities and events that can enhance the user's wellness journey. This storage system plays a critical role in ensuring that the platform can continuously adapt to user needs and provide up-to-date, relevant recommendations throughout the wellness journey.

The user device 102 may interface with the wellness management platform 100 through a synchronized interface. The user device may include a wellness app 114, which serves as the primary platform interface for users. This app allows users to engage with the wellness management system, track their progress, and receive personalized suggestions. The display 115 on the user device provides real-time recommendations 116, suggestions, and alerts generated by the AI module, ensuring users are continuously informed and supported throughout their wellness journey.

This integrated system enables seamless communication between the computing device and the user device, allowing for real-time updates, interactions, and adjustments to the user's wellness plan. Whether through personalized wellness journeys, community-driven support, or real-time recommendations, the platform is designed to provide a comprehensive and adaptive wellness management experience.

Still referring to FIG. 1., the database 110 is configured to store wellness data on a plurality of users enrolled in the wellness management platform 100. Each enrolled user may be pursuing a personalized wellness journey generated by the wellness management platform 100 to achieve at least one wellness goal, with the at least one wellness goal for each user having a progress status associated therewith that is being tracked and updated by the wellness management platform 100 based on accomplishments of each user.

The computing device 101 may be configured to enroll a new user in the wellness management platform, wherein the new user selects at least one wellness goal from a plurality of wellness dimensions.

A personalized wellness journey is generated for the new user, with the personalized wellness journey providing recommendations to achieve the selected at least one wellness goal. Progress of the new user on their personalized wellness journey is tracked based on accomplishments of the new user.

The tracked progress of the new user on their personalized wellness journey is compared to the wellness data stored in the database to identify a convergence with at least one of the users participating in the wellness management platform, wherein the identified convergence corresponds to overlapping or complementary wellness goals between the new user and the at least one user associated with the convergence.

A suggestion is generated for the new user based on the identified convergence. The suggestion includes at least one recommendation for joint activities, shared resources or communications with the at least one user associated with the identified convergence.

The generated suggestion is communicated to the new user over a communications channel, and user options are provided via the generated suggestion to allow the new user to accept, modify, or decline the generated suggestion.

The virtual advisor 107 is to assist the new user in selecting the at least one wellness goal, to define wellness objectives, and to input relevant data for generation of the personalized wellness journey. The virtual advisor may be configured as a chatbot, or a lifelike AI-generated avatar, that uses natural language processing to assist the new user.

As noted above, the AI module 103 is configured to generate the personalized wellness journey, track progress of the new user on their personalized wellness journey, identify the convergence, and generate the suggestion for the new user based on the identified convergence. The AI module may be further configured to make adjustments in the personalized wellness journey based on real-time feedback and progress indicators from the new user.

The wellness dimensions may include mental wellness, physical wellness, spiritual wellness, financial wellness, educational wellness and social wellness. The recommendation for the new user may further include activities for the new user to perform for each selected goal within one or more of the wellness dimensions. The AI module allows the user to set specific timeframes for achieving each selected goal within each wellness dimension, and dynamically adjusts the recommended activities based on the selected timeframes. Notifications related to the personalized wellness journey of the new user are generated by the AI module, with the notifications comprising at least one of reminders, motivational messages, and progress updates.

The generated suggestion for the new user based on the identified convergence may further include recommendations for one or more wellness related activities or events. In other embodiments, the tracked progress of the new user on their personalized wellness journey may be first compared to the available wellness related activities or events to identify one or more wellness related activities or events that may be beneficial to the new user. This comparing may be performed prior to identifying a convergence with at least one of the users participating in the wellness management platform.

FIG. 1.1 illustrates detailed architecture and components of the wellness management platform, integrating multiple modules and interfaces that interact with external applications and services. The figure provides a comprehensive view of the platform's functionality, including data flow, user interaction, and feedback mechanisms.

Starting with the platform interface 150, this section includes key components like the user interface 151, administration portal 152, and analytics interface 153. These interfaces enable different user roles to interact with the platform, access data, manage wellness journeys, and analyze performance metrics.

The application interfaces (API) 154 connect the platform to external apps and services 155, such as third-party applications or services that provide additional wellness-related data. This external data, represented by a cloud icon, may include health alerts, geolocation data, or other wellness-related information that the platform may use to enhance user experience.

The process and procedure engine 156 serves as the core automation component of the platform, designed to streamline and automate documented processes and procedures within an organization. By leveraging user-specific data, the engine dynamically determines the most efficient way to navigate complex workflows and administrative tasks.

Additionally, the engine may include a predictive guidance system 157, which anticipates the next steps in a process, providing users with insights on what to expect and how to prepare for upcoming tasks. This ensures that users are not only informed but are also actively guided through each stage of their wellness journey or administrative process, improving user engagement and efficiency.

The process and procedure engine powers the platform's functionality and includes the interactivity module 158, which features components such as the virtual advisor 159, interactive learning courses 160, and tutorials 161. These tools support user engagement by facilitating goal setting, progress monitoring, and offering customization options. The AI module 162 within the engine is responsible for convergence detection 163 and generating personalized wellness journeys 164, providing users with personalized recommendations and dynamically adjusting their wellness paths based on real-time data.

Data is stored in the data storage 165 section, which houses various types of data that are crucial for the platform's operations and intelligence. This section includes aggregate user data 166, which is a compilation of anonymized data collected from all users of the platform. Examples of aggregate data include trends in wellness activities, common health goals, and average progress metrics.

By analyzing this data, the platform can identify broader trends and make more informed recommendations across its user base. For instance, if the platform detects that a significant portion of users are focusing on improving mental wellness during a particular season, it may adjust its recommendations for new users accordingly.

Clinician data 167 comprises input from healthcare professionals, such as treatment plans, medical advice, and health metrics provided during clinical visits. This data enhances the platform's intelligence by allowing it to integrate professional medical insights into the wellness journeys. For example, if a clinician recommends a specific diet or exercise regimen for a user, the platform can incorporate these recommendations into the user's personalized wellness journey, ensuring that the user's medical needs are aligned with their wellness goals.

Meta data 168 refers to contextual information that helps organize and interpret other data types within the platform. This may include metadata about the time and location of user activities, the types of devices used to access the platform, and the frequency of interactions. For example, the platform may use metadata to determine the most effective times to send wellness reminders based on when the user typically engages with the platform. By leveraging metadata, the platform can refine its algorithms to deliver more contextually relevant recommendations, improving overall user engagement.

User-specific data 169 is individualized information that the platform collects from each user, including their preferences, goals, and interaction history. This data allows the platform to create highly personalized wellness journeys fitted to each user's unique needs and circumstances. For example, if a user consistently engages with content related to physical fitness but shows little interest in mental wellness topics, the platform can prioritize fitness-related recommendations while still subtly encouraging mental wellness activities.

Finally, user health records 170 are a critical component of user-specific data that includes detailed health information such as medical history, current medications, chronic conditions, and recent health metrics. By integrating this data, the platform can ensure that its recommendations are medically appropriate and aligned with the user's overall health status. For instance, if a user has a history of cardiovascular issues, the platform may adjust fitness recommendations to include low-impact exercises that are safer for their condition. Similarly, if the user's health record indicates a need for regular blood pressure monitoring, the platform can remind the user to perform these checks and log the results.

The platform includes several feedback loops 171, critical for continuous improvement and user satisfaction. These loops gather information such as user satisfaction, health data updates, and progress reports to refine the platform's algorithms and improve service quality.

While the figure provides an overview of the wellness management platform's architecture, it should be understood that additional modules, features, or data categories may be integrated as the platform evolves. These could include new data sources, third-party integrations, or expanded user interaction mechanisms to further enhance the platform's adaptability and functionality.

FIG. 1.2 illustrates an embodiment of the user interface and initial onboarding process of the wellness platform, which may guide users through setup, data input, and goal-setting phases. In accordance with various embodiments of the present disclosure, the onboarding process may be facilitated by an Al-powered virtual advisor, which may interact with users and ensure a seamless introduction to the platform's features.

The user interface (UI) 173 may serve as the main access point for users to engage with the wellness platform. It may be designed to be intuitive and accessible, featuring a clean and minimalistic design that supports multiple languages. The UI may be optimized for both mobile devices and desktop applications, providing flexibility and case of use across different platforms.

In some embodiments, the onboarding process may be facilitated by a lifelike AI-generated avatar 174 or AI-powered chatbot, which may interact with users to collect necessary information and guide them through the initial setup. The avatar may support multiple languages 175, making it accessible to a diverse user base. It may be equipped with advanced natural language processing (NLP) capabilities, allowing it to effectively understand and respond to user queries and inputs.

The initial setup screen 176 may be the first interface presented to the user. Here, the avatar 174 may welcome the user and begin the onboarding process by asking a series of introductory questions designed to gather basic user information, such as name, age, and general wellness goals.

Following the initial setup, users may be directed to a data input interface 177, where they can provide detailed personal information. This interface may allow users to input a wide range of data, including health metrics, lifestyle preferences, and wellness goals. The data input fields 178 may be dynamically generated based on user responses, ensuring that the information collected is relevant and comprehensive.

In other embodiments, the user may be guided to a goal-setting module 179 after providing the necessary data. Here, users may define specific wellness objectives across various dimensions, such as mental, physical, spiritual, financial, educational, and social well-being.

The module may include predefined goal templates 180 as well as options for users to create custom goals 181 that meet their unique needs. The progress overview screen 182 may provide users with a summary of their current wellness status based on the data they have inputted. This screen may display initial insights and suggestions generated by the AI, offering users a glimpse of their personalized wellness journey. The overview may include visual indicators 183, such as progress bars or charts, to help users track their goals.

The onboarding process may include a feedback loop 184, where users can provide immediate feedback on their experience with the virtual advisor and the platform's interface. This feedback may be processed by the AI to refine future interactions and improve the overall user experience.

The user interface may also include an easily accessible help and support section 185, where users can find answers to common questions, access tutorials, or contact customer support for additional assistance. To ensure seamless access across different devices, the platform may include a multi-platform synchronization feature. This feature may allow users to start their onboarding process on one device and continue it on another without losing any progress.

FIG. 1.3 illustrates a real-time interaction scenario within the wellness management platform, focusing on Bluetooth-connected devices. The diagram shows how wearable devices, such as smartwatches, fitness trackers, sleep trackers, stress trackers, and health trackers for respiratory rate and blood oxygen levels, may be paired with the user's mobile device. The central element in the figure is a smartphone interface that prompts the user with the option to pair their fitness tracker. The user may provide a response by selecting either yes or no, enhancing case of use.

Additionally, the figure represents health monitoring devices that may include blood glucose monitors, blood pressure monitors, digital scales, pulse oximeters, insulin pumps, cardiac monitoring systems, and epilepsy management devices. These devices may also be connected to the smartphone via Bluetooth. This configuration allows the platform to receive real-time health metrics from multiple sources simultaneously, contributing to the dynamic wellness journey described in the specification. These integrations may provide a comprehensive view of the user's wellness. By allowing the AI-driven system to continuously monitor and adapt the user's wellness journey based on real-time data inputs from these devices, the platform supports dynamic, personalized wellness management.

FIG. 1.4 illustrates real-time interaction scenarios within the wellness management platform, focusing on locally installed applications on the user's device. The central element in this figure is a smartphone interface prompting the user to decide whether to be reminded of their medical appointments. The user may provide input by selecting yes or no, which facilitates user control over the interactions with the platform.

The figure depicts various applications that may interface with the platform, such as appointment schedulers, nutrition tracking apps, sleep quality apps, and mental health apps. These applications may provide vital data that the wellness platform utilizes to create and adapt personalized wellness journeys. For example, appointment schedulers may help users manage their medical or wellness-related appointments, while nutrition tracking apps could provide insights into dietary habits that influence physical and mental well-being. Sleep quality apps may offer data on sleep patterns, and mental health apps may contribute information related to mood tracking, therapy sessions, or other mental health interventions.

The application interfaces represented in this figure suggest that these locally installed apps communicate with the platform in real-time, continuously feeding data into the system. This interaction may allow the platform's AI-driven engine to process the data and update recommendations and wellness paths accordingly, ensuring that the user's journey is dynamic and reflective of their real-time circumstances. The figure emphasizes the integration of multiple data sources within the user's device, which may enhance the platform's ability to provide comprehensive and personalized wellness management.

FIG. 1.5 illustrates a real-time interaction scenario within the wellness management platform, focusing on web-based connections to external data sources and services. The central element in this figure is a smartphone interface prompting the user to decide whether to connect to local events that can help achieve their wellness goals. The user may provide input by selecting yes or no, which facilitates user control over the interactions with the platform.

The figure showcases various cloud-based data sources and services connected to the platform through web-based connections. These include geolocation data, community events, health alerts, social media, online wellness content, virtual fitness classes, medical appointments, and financial health indicators. Each of these data sources represents a web-based service that may influence the user's wellness journey.

For example, geolocation data may provide real-time information about the user's location, which the platform may use to suggest relevant local events or services. Community events may include wellness-related activities such as workshops, seminars, or support groups that align with the user's goals. Health alerts could provide important notifications about public health concerns or individual health risks. Social media may allow the platform to incorporate social connections and support networks into the user's wellness journey.

Online wellness content could include articles, videos, or courses that provide valuable information or motivation. Virtual fitness classes may offer users opportunities to engage in physical activities that support their wellness objectives. Medical appointments may help track and manage healthcare interactions, while financial health indicators could offer insights into how financial well-being impacts overall wellness.

The web-based connections illustrated in this figure emphasize the platform's ability to integrate external data and services in real time, ensuring that the user's wellness journey is continuously updated and relevant. This interaction may enable the system's AI-driven engine to process the incoming data and update recommendations and wellness paths accordingly, ensuring that the user's journey is dynamic and reflective of their real-time circumstances. The figure highlights the importance of integrating multiple external data sources and services, which may enhance the platform's ability to provide comprehensive and personalized wellness management.

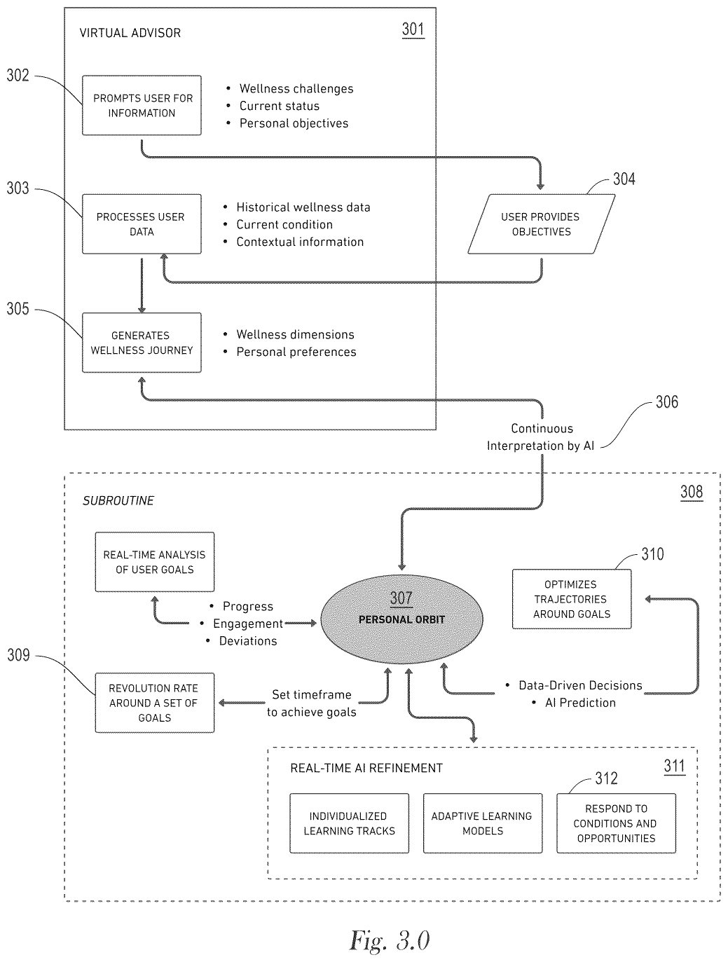

FIG. 2.0 illustrates a virtual advisor system 200 in accordance with an embodiment of the present disclosure, focusing on the process of guiding users through the creation and management of their personalized wellness journeys. The virtual advisor system 200 facilitates a dynamic and responsive wellness plan based on user input and AI-driven analysis, ensuring that each user's wellness journey is customized to their unique needs.

The process begins with the user defining their wellness objectives 201. These objectives may span multiple dimensions of wellness, including mental, physical, spiritual, financial, educational, and social aspects. The virtual advisor assists the user in specifying their goals, ensuring that the user's intentions are clearly defined. Once the wellness objectives are established, the user provides additional input 202, which may include motivations, rationales, and other relevant personal data. This step helps the AI engine 203 better understand the user's priorities and the context of their wellness journey.

The Al engine 203 processes the user's input and performs a database query 204 to gather relevant data. During this analysis, the AI engine considers interdependencies between the defined objectives 205, ensuring that the wellness paths generated are holistic and interconnected. This step is crucial for creating a comprehensive set of wellness paths 206 that align with the user's goals. Based on the AI's analysis, a set of wellness paths 206 are formulated. These paths serve as a structured guide for the user, addressing both immediate and long-term goals. The user then selects specific goals 207 from the set of wellness paths, allowing them to focus on particular aspects of their wellness journey.

At this stage, the user may determine a set time 208 to achieve their goals, deciding on the timeframe within which they want to complete their wellness objectives. This decision involves setting the “revolution rate”, which indicates the speed at which the user intends to progress through their wellness journey. After determining the timeframe, if any, the AI engine crafts the wellness journey 210. This journey is a personalized plan that outlines the steps and milestones the user will follow to achieve their objectives. The wellness journey is organized into an actionable plan 211, which includes multiple learning tracks. These tracks may focus on different aspects of the user's goals, providing structured guidance and activities to support their progress.

The user reviews and approves the action plan 212, ensuring that it aligns with their expectations and preferences. After approval, the platform engages with the user 213, initiating the wellness journey and providing ongoing support and guidance. Throughout the journey, feedback and insights are dispatched 214 to the user, allowing them to stay informed about their progress and receive personalized recommendations.

A feedback loop 215 is established, enabling the AI engine to continuously monitor the user's progress and gather feedback. Based on the feedback received, the AI engine adjusts the wellness journey 216 to ensure that it remains relevant and effective as the user progresses. The virtual advisor continues to provide digital guidance 217 throughout the wellness journey, offering personalized advice and support based on the user's evolving needs. The feedback and updates are delivered to the user through various channels, including app notifications, emails, and SMS, ensuring that the user remains connected to their wellness journey at all times.

The AI engine also continuously monitors the user's progress 219, making real-time adjustments to the plan as necessary. This ensures that the wellness journey is responsive to both the user's actions and any external factors that may influence their progress.

Although not depicted in the drawing, the virtual advisor system 200 may include the collection and integration of additional data types, such as biometric data, psychological assessments, lifestyle preferences, and social interactions. The platform may also incorporate advanced user engagement features such as personalized reminders, detailed progress tracking, gamified milestones, and interactive feedback tools. These features further enhance the user experience by providing a more comprehensive and responsive wellness journey adapted to individual needs and preferences.

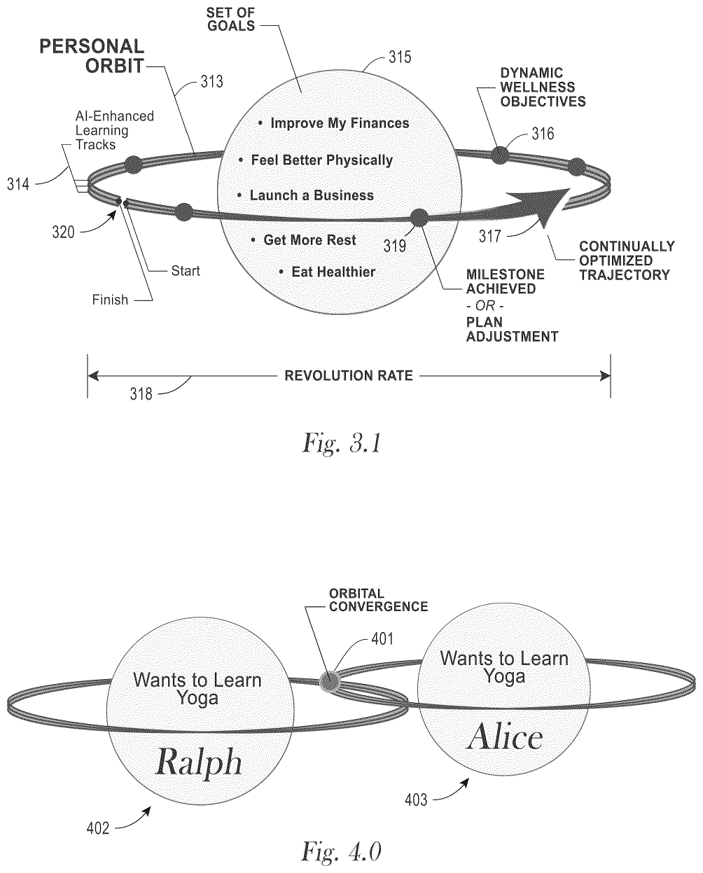

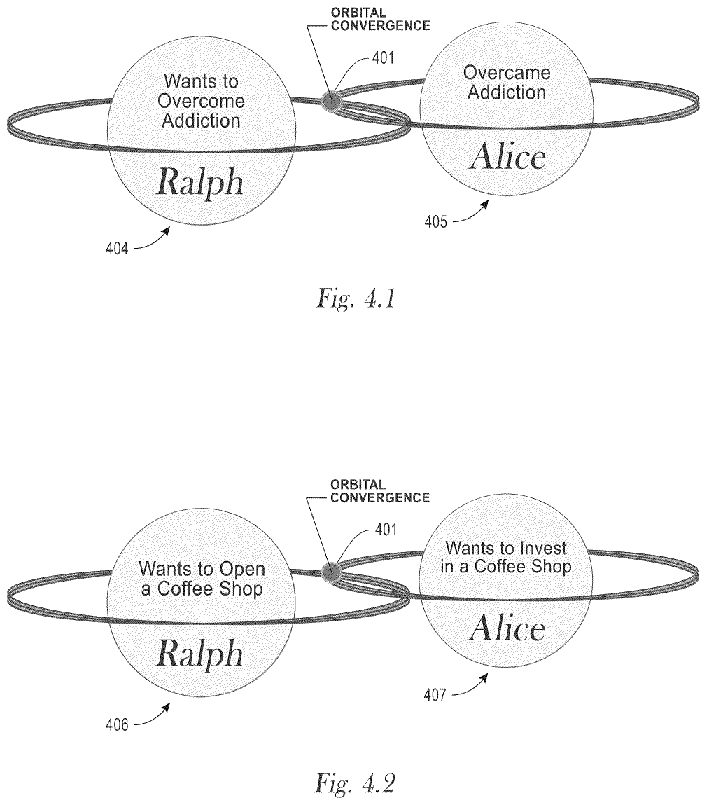

FIG. 3.0 illustrates the concept of a “Personal Orbit” within the wellness journey, managed by an AI-enhanced virtual advisor, in accordance with an embodiment of the present disclosure. This figure highlights the dynamic interaction between the user and the AI system, which generates, monitors, and adjusts a personalized wellness trajectory to ensure continuous alignment with the user's evolving objectives and circumstances. The virtual advisor 301 interacts with the user, analyzes data, and generates customized wellness journeys based on the inputs received.

The process begins with the virtual advisor, which prompts the user for information 302 such as wellness challenges, current status, and personal objectives. This information gathering is crucial for understanding the user's starting point and desired outcomes. The virtual advisor processes user data 303, which includes historical wellness data, current conditions, and contextual information. This data processing step allows the system to gain a comprehensive understanding of the user's situation and personalize the wellness journey accordingly.

The user provides their objectives 304, which serve as the foundation for the personalized wellness journey. These objectives may encompass various wellness goals, such as “become more physically fit,” “improve financial health,” or “discover a new hobby.” The virtual advisor processes the user's data by synthesizing the information provided by the user with global data stored in the system's database, such as historical wellness data and contextual information. Based on this comprehensive analysis, the virtual advisor generates a wellness journey 305 that aligns with the user's goals and personal preferences. The system takes these specific goals into account and creates a personalized plan that supports the user in achieving their desired outcomes through a structured and dynamic process.

The continuous interpretation by AI 306 ensures that the system remains responsive to the user's progress and any changes in their situation. This real-time analysis is critical for maintaining the relevance and effectiveness of the wellness journey. The concept of a personal orbit 307 represents the user's ongoing progress within the wellness journey. The personal orbit is a dynamic model that the AI interprets in real-time, considering factors such as progress, engagement, and deviations from the expected path.

A subroutine 308 supports the personal orbit by performing real-time analysis of user goals. This subroutine monitors progress, engagement, and any deviations, feeding this information back into the system for further refinement. The revolution rate 309, which reflects the timeframe within which the user aims to achieve their goals, is an optional input that influences how the personal orbit evolves. Users may set specific timeframes to complete their wellness journey, which directly affects the pace at which the AI adjusts their wellness plan.

The AI continuously optimizes trajectories around goals 310 by making data-driven decisions and predictions. This optimization process ensures that the user's wellness journey remains aligned with their objectives and adapts to any changes in their circumstances. Real-time AI refinement 311 enables the system to adjust individualized learning tracks and adaptive learning models. This refinement allows the system to respond to conditions and opportunities 312 as they arise, ensuring that the user receives the most relevant and effective guidance.

The system's real-time responsiveness is further enhanced by its ability to incorporate external factors, such as emerging wellness trends or unexpected life events, into the user's personal orbit. The system adapts to these conditions by modifying the user's wellness journey in real-time, ensuring continuous progress toward their goals. Although not depicted in the drawing, the virtual advisor system 301 may also include features that allow users to provide feedback on their wellness journey. This feedback can be collected through surveys, direct input, or passive engagement metrics, and is used by the AI to refine the system's recommendations and ensure that the user's experience is as personalized and effective as possible. Additionally, the system may include alerts for critical health updates or external events that could impact the user's wellness journey, further enhancing its real-time responsiveness.

FIG. 3.1 illustrates the Personal Orbit in greater detail, showing how a user's wellness journey is structured around specific goals and dynamically adjusted by the AI engine, in accordance with an embodiment of the present disclosure. This figure expands on the concept introduced in FIG. 3.0, emphasizing the continuous nature of the wellness journey and how it evolves over time based on user progress and external factors.

The central component of the diagram is the Personal Orbit 313, which represents the path that the user follows in pursuit of their wellness goals. This orbit is continuously shaped by the Al to optimize the user's progress. The orbit is composed of multiple AI-enhanced learning tracks 314. Each track is adapted to specific wellness objectives and guides the user through actionable steps that contribute to their overall journey. At the core of the orbit is the user's set of goals 315. These goals are personalized and may vary based on the user's individual preferences and needs.

As the user progresses, the AI introduces dynamic wellness objectives 316 along the orbit. These objectives are adaptable and may change in response to the user's progress, emerging opportunities, or external factors.

The AI is responsible for maintaining a continually optimized trajectory 317. This involves analyzing the user's performance and adjusting the path to ensure that the wellness journey remains aligned with the user's goals and timeframes. The “Revolution Rate” 318 indicates the speed at which the user moves through their wellness journey. This rate may be adjusted based on the user's preferences, the urgency of their goals, or recommendations from the Al. For instance, a user might choose a faster revolution rate for achieving short-term goals or a slower pace for long-term objectives.

Along the orbit, key milestones are marked 319. Upon reaching these milestones, the Al evaluates whether the user has achieved their goals or if a plan adjustment is necessary. This process ensures that the user's journey remains relevant and effective.

The start and finish points 320 represent the user-defined beginning and end dates for completing a specific wellness journey. These dates generally define the length of a single revolution rate, which is the time it takes for the user to complete one full cycle of their wellness orbit. The ability for a user to establish a specific timeframe for their revolution rate is optional and may be adjusted based on individual preferences or the recommendations of the AI. This flexibility allows users to set a pace that aligns with their personal goals, whether they prefer a rapid progression or a more gradual approach.

Although not depicted in the drawing, the system may also account for additional context by integrating external data sources such as global wellness trends, new research findings, or user-specific data like real-time health metrics. This integration could further refine the dynamic wellness objectives and the continually optimized trajectory. Moreover, the system might include variability in revolution rates, allowing users to set different paces for different types of goals based on their current life circumstances or priorities.

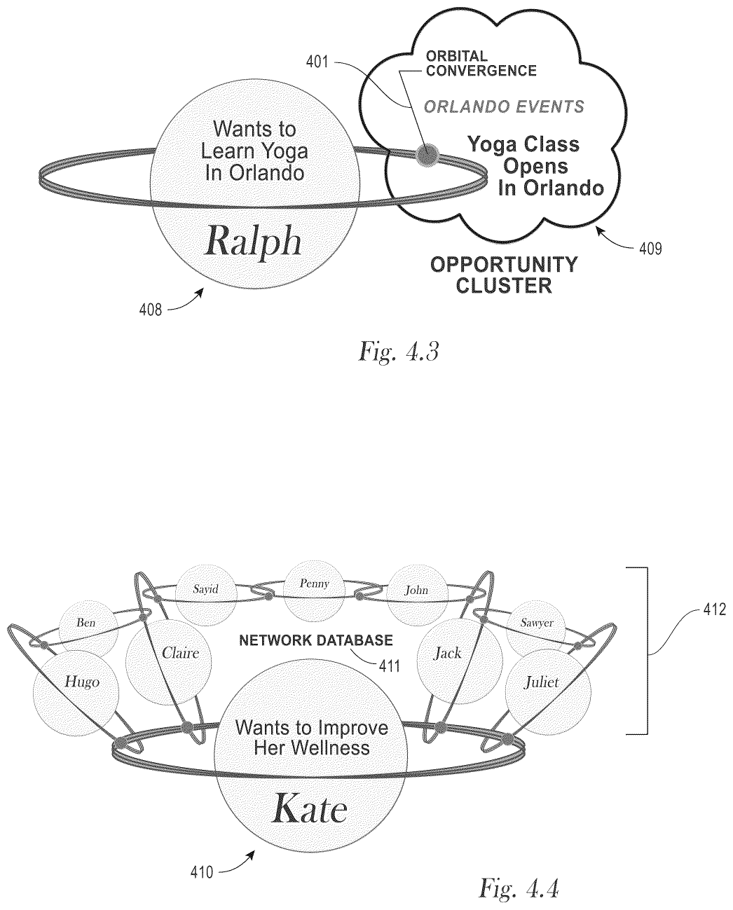

FIG. 4.0 illustrates the concept of “Orbital Convergence” within the wellness platform, where the wellness journeys of different users intersect based on shared goals. The system detects these intersections, known as Orbital Convergences 401, and may utilize this information to suggest collaborative activities, shared resources, or joint progress tracking. For example, as depicted, upon detecting an Orbital Convergence, the platform may recommend a joint yoga session, provide access to shared instructional materials, or suggest peer support activities that enhance the experience for both users.

User Ralph 402 is depicted with a wellness journey focused on learning yoga. The system tracks his progress along a defined path or orbit. User Alice 403 similarly has a goal of learning yoga, with her wellness journey also tracked by the system. The Orbital Convergence point 401 represents the intersection where Ralph's and Alice's wellness journeys align, prompting the system to recognize the potential for collaboration.

Although not depicted in the drawing, the platform may extend this concept to other wellness goals beyond yoga, such as fitness, diet, or mental wellness. In such cases, the system could facilitate convergences around different activities, such as group fitness challenges, shared meal planning, or joint meditation sessions. Moreover, users may have the option to opt in or out of these collaborative opportunities, depending on their preferences, allowing for a personalized and flexible experience.

The platform's versatility is further demonstrated by its ability to identify Orbital Convergences in various contexts. For instance, users focusing on financial literacy might experience a convergence when they set similar savings goals, leading the system to suggest shared financial planning resources or group investment discussions.

The platform may also incorporate user feedback to refine its suggestions around Orbital Convergence 401. For example, if users prefer solo activities, the platform could adjust future recommendations accordingly, ensuring the convergence feature aligns with individual user preferences and enhances their overall wellness journey.

FIG. 4.1 illustrates an example of Orbital Convergence 401 within the wellness platform, specifically showcasing how two users, Ralph 404 and Alice 405, experience an intersection of their wellness journeys. In this embodiment, Ralph's objective is to overcome addiction, while Alice has already achieved this goal. The platform detects this overlap and identifies an opportunity for Ralph and Alice to connect based on their shared focus.

Ralph's orbit is defined by his specific wellness objective of overcoming addiction, while Alice's orbit represents her successful journey in the same area. The platform facilitates this connection through Orbital Convergence 401, where the two orbits intersect. Upon detecting this convergence, the platform may suggest various actions, such as initiating a mentorship opportunity between Ralph and Alice, recommending shared support group sessions, or enabling direct communication between the two users to foster mutual support and guidance.

Although not depicted in the drawing, this connection may be further customized by the platform, allowing users to opt-in or out of these suggested interactions, providing them with control over their engagement. The platform may also expand this feature to other areas of wellness, such as mental health or career goals, where users' objectives align in meaningful ways.

FIG. 4.2 illustrates an instance of Orbital Convergence 401 within the wellness platform, specifically demonstrating how two users, Ralph 406 and Alice 407, experience an intersection of their wellness journeys centered around a business goal. In this embodiment, Ralph's objective is to open a coffee shop, while Alice's objective is to invest in a coffee shop. The platform detects this overlap and identifies an opportunity for Ralph and Alice to connect based on their complementary goals. Ralph's orbit is defined by his specific business objective of starting a coffee shop, while Alice's orbit represents her interest in investing in such a venture.

The platform facilitates this connection through Orbital Convergence 401, where the two orbits intersect. Upon detecting this convergence, the platform may suggest various actions, such as proposing a potential business partnership, offering investment opportunities, or initiating a connection between Ralph and Alice for further discussions regarding their shared interest.

Although not depicted in the drawing, this connection may be further customized by the platform, allowing users to opt-in or out of these suggested interactions, providing them with control over their engagement. The platform may also extend this feature to other business contexts, such as shared entrepreneurial goals or networking opportunities, where users' objectives align in meaningful ways.

FIG. 4.3 illustrates an instance of Orbital Convergence 401 within the wellness platform, where Ralph 408 experiences a convergence between his goal to learn yoga in Orlando and an external “Opportunity Cluster” 409. The Opportunity Cluster is a broader intersection point that diverges from the specific interaction between individual users and instead encompasses a range of relevant opportunities, such as events happening within a community or interactions among subsets of users that align with Ralph's wellness objectives.

Ralph's orbit, centered around his desire to learn yoga, intersects with this Opportunity Cluster 409 where the platform identifies an alignment between his goal and an upcoming yoga class opening in Orlando. This broader cluster signifies not just a single point of intersection but a collection of related opportunities that the platform may present to Ralph.

Upon detecting this convergence, the platform may notify Ralph of this opportunity through various channels, such as app notifications, emails, or SMS, enabling him to take advantage of the event. The platform might also suggest additional related opportunities within the cluster, such as workshops, seminars, or other local wellness events, further enriching Ralph's wellness journey.

Although not depicted in the drawing, the platform may offer users the option to opt-in for such notifications, granting control over which types of opportunities they wish to be informed about. This broader approach to identifying Opportunity Clusters demonstrates the platform's versatility in connecting users not just with each other but also with external events and resources that support their holistic wellness goals.

FIG. 4.4 illustrates the concept of a network-driven Orbital Convergence within the wellness platform, where Kate 410 seeks to improve her wellness. This figure demonstrates how the platform's network database 411 identifies potential synergies among a collection of various individuals 412 who may share similar wellness goals or activities. The platform leverages these connections to facilitate group-based wellness activities or goals.

In this example, Kate's wellness journey intersects with those of other users within the network, leading to possible group activities such as shared fitness challenges, group therapy sessions, or wellness workshops. The network database 411 plays a pivotal role in identifying these convergences by analyzing user data and recognizing overlapping objectives or interests.

Upon detecting such convergences, the platform may present Kate and the other users with opportunities to engage in collective wellness activities, thus enhancing their individual and group wellness experiences. The platform may notify users of these opportunities through various channels, such as app notifications, emails, or SMS. The platform may also allow users to have control over their participation in these network-driven convergences. For example, users like Kate can choose to opt into certain group activities or decline others based on their preferences, ensuring that the platform respects user autonomy and comfort levels in social interactions.

Although not depicted in the drawing, the network database 411 may integrate real-time data or feedback from users to continuously refine and optimize these group-based wellness interactions. This ensures that the convergences remain relevant and beneficial to all participants, demonstrating the platform's dynamic and user-centered approach to holistic wellness.

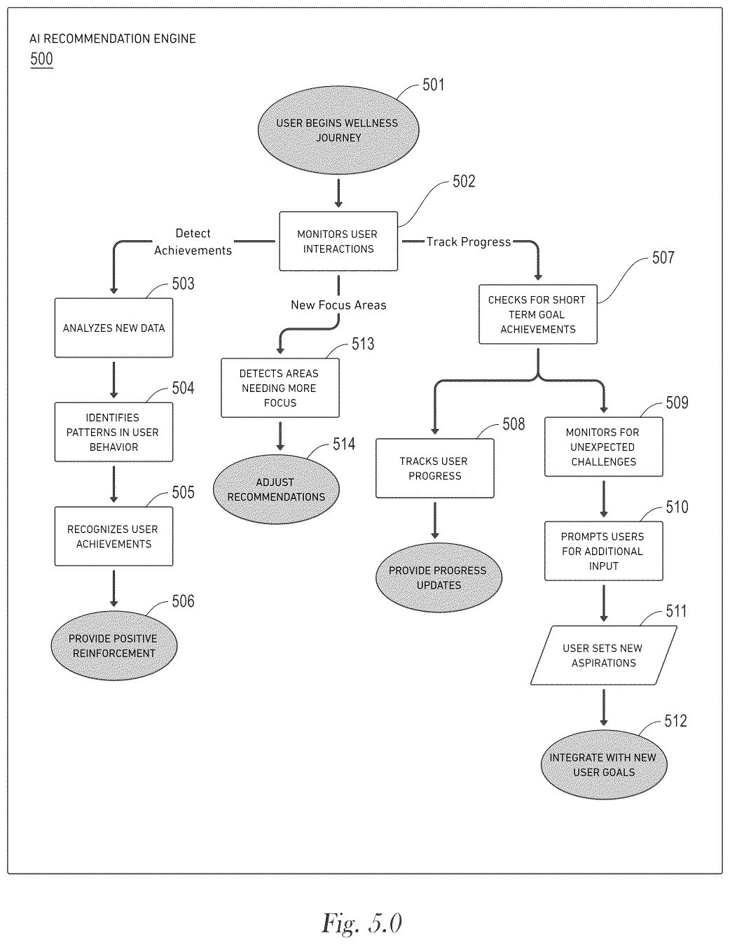

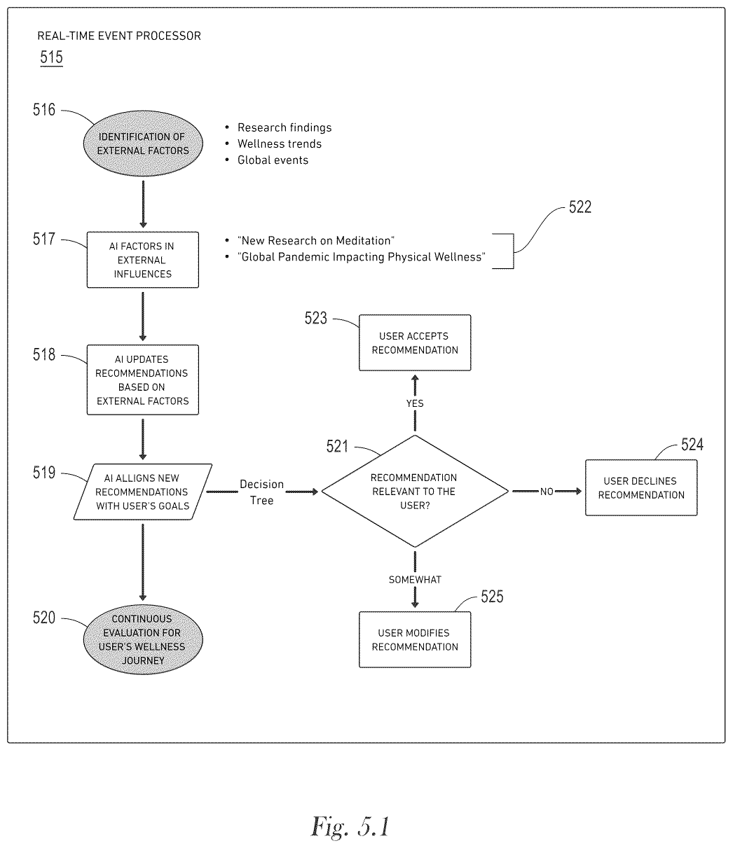

FIG. 5.0 illustrates an AI recommendation engine 500 within the wellness management platform. The engine's interaction with the user starts at the initiation of the user's wellness journey 501, where the platform begins monitoring user interactions 502. This is a continuous process, ensuring that the platform detects achievements and tracks the user's progress and activities.

The platform enters a phase of data analysis 503, where the AI system processes new data inputs generated from the user's interactions. This analysis is aimed at identifying patterns in user behavior 504, such as recurring actions, preferences, or challenges. Upon identifying patterns, the AI system is equipped to recognize user achievements 505, providing positive reinforcement and feedback 506.

Simultaneously, the AI checks for short-term goal achievement 507 to ensure that users are on track with their objectives. This process includes tracking the user's progress 508 and monitoring for unexpected challenges 509, which may arise during their wellness journey.

When unexpected challenges are detected, the AI system may prompt the user for additional input or decisions 510, showcasing the platform's interactive nature. In the event new aspirations are set by the user 511, the platform adapts by integrating these new goals into the ongoing analysis 512.

The system also detects areas that require more focus 513, ensuring that no critical aspects of the user's wellness journey are overlooked. Following this, the AI adjusts its recommendations 514 based on the identified needs and patterns, customizing the user's experience to be as effective and personalized as possible.

Although not depicted in the diagram, the platform may include a feedback loop that integrates user satisfaction and feedback into the system, influencing future adjustments and emphasizing the platform's adaptability. Additionally, key decision points may exist where the AI prompts the user for further input or allows manual adjustments, ensuring that users maintain autonomy over their wellness journey.

Moreover, the system is designed with flexibility in mind, allowing users to manually adjust or override Al-generated recommendations, providing a higher degree of control over their journey. This flexibility ensures that the platform can accommodate a wide range of user preferences and needs.

The process illustrated in FIG. 5.1 can be further understood as a function of the platform's real-time event processor 515, which leverages advanced AI algorithms to manage and analyze incoming data streams in real time. This allows the system to swiftly process up-to-the-minute information and make real-time contextual inferences on a large amount of data. The process begins with the identification of external factors 516, such as new research findings, emerging wellness trends, or significant global events. These external factors are crucial as they influence the context in which the user's wellness journey unfolds. The platform's AI engine, functioning as part of an event-driven architecture, captures these real-time events and converts them into appropriate embeddings. This allows the AI system to apply just-in-time logic, ensuring that its recommendations remain timely and relevant.

Incorporating real-time media monitoring and real-time processing, the AI engine factors in these external influences 517 and updates its recommendations accordingly 518. Data streaming plays a pivotal role here, enabling the AI to continuously process and integrate new information, ensuring that the wellness paths remain relevant and beneficial even as the broader wellness landscape evolves. Once the AI updates its recommendations based on the external factors, it ensures that these recommendations align with the user's specific journey and the broader wellness landscape 519. This alignment is essential to maintain the relevance and effectiveness of the wellness paths the user is following.

The process concludes with the platform ensuring continuous evaluation 520 of the user's wellness journey. This continuous evaluation process reflects the platform's adaptability and responsiveness to both user data and external influences, making the wellness journey more personalized and effective over time. Additionally, the platform's real-time event processor enhances the Al's ability to react promptly to changes, further fine-tuning its recommendations.

To enhance user experience, the platform may include a feedback mechanism 521 where the Al can receive real-time data from users about the relevance of the updated recommendations. This feedback could further improve the Al's ability to adapt and refine its suggestions, ensuring that the user's wellness journey remains aligned with their evolving needs.

For instance, specific examples of external factors 522 such as “New Research on Meditation” or “Global Pandemic Impacting Physical Wellness” could provide more relatable context to the users, showcasing how the AI integrates such data into its recommendations.

Furthermore, users may have the ability to accept 523, decline 524, or modify 525 these AI-updated recommendations based on their preferences. This feature emphasizes user autonomy within the platform, allowing individuals to maintain control over their wellness journey while benefiting from the Al's guidance.

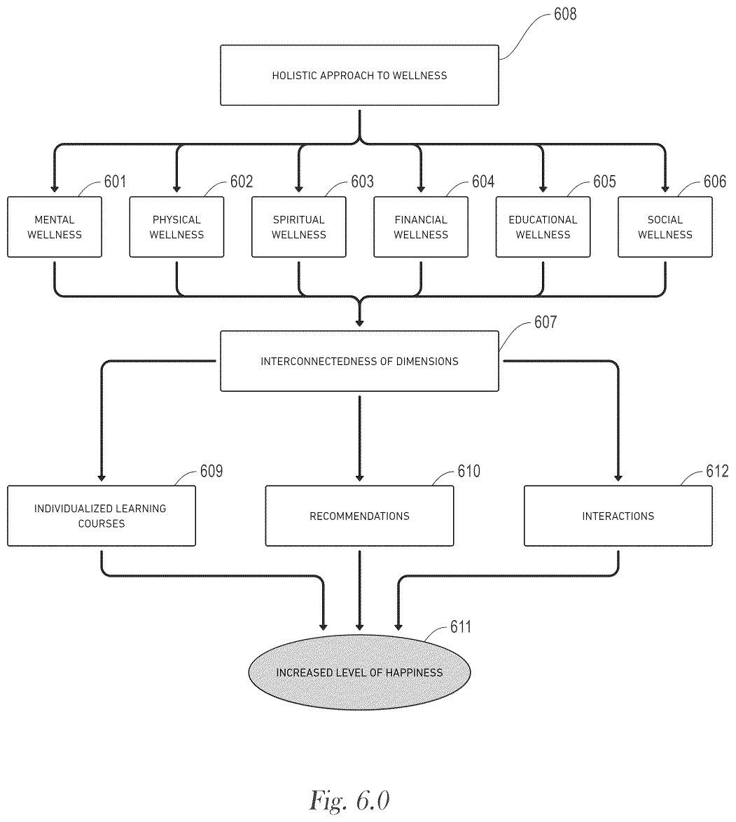

FIG. 6.0 illustrates the holistic approach to wellness adopted by the platform, which integrates multiple dimensions of wellness to create a comprehensive and interconnected wellness journey for the user. The figure highlights six key wellness dimensions: Mental Wellness 601, Physical Wellness 602, Spiritual Wellness 603, Financial Wellness 604, Educational Wellness 605, and Social Wellness 606. These dimensions are foundational elements that contribute to the user's overall well-being. However, it should be noted that these six dimensions are not limiting, and other key wellness dimensions are envisioned within the scope of the platform.

At the core of this system is the concept of “Interconnectedness of Dimensions” 607, which underscores how each dimension influences and interacts with the others. For example, improvements in one area, such as Financial Wellness 604, might have a positive impact on Mental Wellness 601, and Social Wellness 606, demonstrating the integrated nature of the wellness journey. The “Holistic Approach to Wellness” 608 involves recognizing these interconnections and leveraging them to create a balanced and fulfilling life for the user. The platform uses this holistic approach to customize individualized learning courses 609, which are designed to address the unique needs and goals of the user across the various wellness dimensions.

As the user engages with the platform, they receive recommendations 610 that are informed by the Al's analysis of their data and progress. These recommendations are intended to guide the user in making decisions that align with their wellness objectives and to help them achieve an “Increased Level of Happiness” 611. User interactions 612, play a critical role in shaping the wellness journey. The platform may facilitate interactions that are beneficial to the user's wellness, such as connecting them with others who share similar goals or providing access to community resources that support their wellness journey.

Although not depicted in the diagram, the platform may include feedback loops where the user's progress or interactions influence the wellness dimensions or recommendations, emphasizing the dynamic nature of the wellness journey. For instance, as the user makes progress in Physical Wellness 602, the platform might adjust its recommendations to focus more on Spiritual Wellness 603 or Educational Wellness 605, ensuring a well-rounded approach. Moreover, users may have the autonomy to prioritize or focus on specific wellness dimensions, allowing the platform to accommodate individual preferences and mold the wellness journey to align with the user's personal goals and values.

Many modifications and other embodiments will come to the mind of one skilled in the art having the benefit of the teachings presented in the foregoing descriptions and the associated drawings. Therefore, it is understood that the foregoing is not to be limited to the example embodiments, and that modifications and other embodiments are intended to be included within the scope of the appended claims.

1. A wellness management platform for facilitating user interactions based on shared wellness goals, comprising:
a database configured to store wellness data on a plurality of users enrolled in the wellness management platform, with each user pursuing a personalized wellness journey generated by the wellness management platform to achieve at least one wellness goal, with the at least one wellness goal for each user having a progress status associated therewith that is being tracked and updated by the wellness management platform based on accomplishments of each user; and
a computing device coupled to the database and configured to perform the following:
enroll a new user in the wellness management platform, wherein the new user selects at least one wellness goal from a plurality of wellness dimensions,
generate a personalized wellness journey for the new user, with the personalized wellness journey providing recommendations to achieve the selected at least one wellness goal,
track progress of the new user on their personalized wellness journey based on accomplishments of the new user,
comparing the tracked progress of the new user on their personalized wellness journey to the wellness data stored in the database to identify a convergence with at least one of the users participating in the wellness management platform, wherein the identified convergence corresponds to overlapping or complementary wellness goals between the new user and the at least one user associated with the convergence,
generate a suggestion for the new user based on the identified convergence, wherein the suggestion includes at least one recommendation for joint activities, shared resources or communications with the at least one user associated with the identified convergence,
communicate the generated suggestion to the new user over a communications channel, and
provide user options via the generated suggestion to allow the new user to accept, modify, or decline the generated suggestion.

2. The wellness management platform of claim 1, wherein the computing device comprises a virtual advisor to assist the new user in selecting the at least one wellness goal, to define wellness objectives, and to input relevant data for generation of the personalized wellness journey.

3. The wellness management platform of claim 2, wherein the virtual advisor comprises a chatbot that uses natural language processing to assist the new user.

4. The wellness management platform of claim 1, wherein the computing device comprises an artificial intelligence (AI) module to generate the personalized wellness journey, track progress of the new user on their personalized wellness journey, identify the convergence, and generate the suggestion for the new user based on the identified convergence.

5. The wellness management platform of claim 4, wherein the AI module is further configured to make adjustments in the personalized wellness journey based on real-time feedback and progress indicators from the new user.

6. The wellness management platform of claim 1, wherein the plurality of wellness dimensions comprise mental wellness, physical wellness, spiritual wellness, financial wellness, educational wellness and social wellness.

7. The wellness management platform of claim 6, wherein the recommendation for the new user further includes activities for the new user to perform for each selected goal within one or more of the wellness dimensions.

8. The wellness management platform of claim 7, wherein the computing device is configured to:
allow the user to set specific timeframes for achieving each selected goal within each wellness dimension; and
dynamically adjust the recommended activities based on the selected timeframes.

9. The wellness management platform of claim 1, wherein the computing device is configured to generate notifications related to the personalized wellness journey of the new user, with the notifications comprising at least one of reminders, motivational messages, and progress updates.

10. The wellness management platform of claim 1, wherein the generated suggestion for the new user based on the identified convergence further includes recommendations for one or more wellness related activities or events.

11. The wellness management platform of claim 1, wherein identifying the convergence is based on at least one of user-defined wellness objectives, historical progress data, user preferences for interaction, and external wellness-related events or opportunities.

12. The wellness management platform of claim 11, wherein the user preferences for interaction includes virtual meetings, shared wellness activities, mentoring or peer support relationships, collaborative learning sessions, and community event participation.

13. The wellness management platform of claim 1, wherein the computing device is configured to prioritize the convergence based on a degree of complementarity between the at least one goal of the new user and the goals of the users in the database, with higher priority given to convergences that are likely to result in mutually beneficial outcomes.

14. The wellness management platform of claim 1, wherein the computing device is configured to track effectiveness of interactions facilitated by the convergence, and to use this data to refine future recommendations and improve accuracy of convergence identification.

15. The wellness management platform of claim 1, comprising an interface to provide a user-controlled option to manually search for potential convergences based on specific criteria, such as location, wellness focus, or desired interaction type.

16. The wellness management platform of claim 1, wherein the generated suggestion is communicated over the communications channel to the new user via one or more of mobile app notifications, emails, and SMS messages.

17. A method for facilitating user interactions based on shared wellness goals, comprising:
storing in a database wellness data on a plurality of users enrolled in a wellness management platform, with each user pursuing a personalized wellness journey generated by the wellness management platform to achieve at least one wellness goal, with the at least one wellness goal for each user having a progress status associated therewith that is being tracked and updated by the wellness management platform based on accomplishments of each user;
enrolling a new user in the wellness management platform, wherein the new user selects at least one wellness goal from a plurality of wellness dimensions;
generating a personalized wellness journey for the new user, with the personalized wellness journey providing recommendations to achieve the selected at least one wellness goal;
tracking progress of the new user on their personalized wellness journey based on accomplishments of the new user;
comparing the tracked progress of the new user on their personalized wellness journey to the wellness data stored in the database to identify a convergence with at least one of the users participating in the wellness management platform, wherein the identified convergence corresponds to overlapping or complementary wellness goals between the new user and the at least one user associated with the convergence;
generating a suggestion for the new user based on the identified convergence, wherein the suggestion includes at least one recommendation for joint activities, shared resources or communications with the at least one user associated with the identified convergence;
communicating the generated suggestion to the new user over a communications channel; and
providing user options via the generated suggestion to allow the new user to accept, modify, or decline the generated suggestion.

18. The method of claim 17, further comprising operating a virtual advisor to assist the new user in selecting the at least one wellness goal, to define wellness objectives, and to input relevant data for generation of the personalized wellness journey.

19. The method of claim 17, further comprising operating an artificial intelligence (AI) module to generate the personalized wellness journey, track progress of the new user on their personalized wellness journey, identify the convergence, and generate the suggestion for the new user based on the identified convergence.

20. The method of claim 17, wherein the plurality of wellness dimensions comprise mental wellness, physical wellness, spiritual wellness, financial wellness, educational wellness and social wellness.

21. The method of claim 20, wherein the recommendation for the new user further includes activities for the new user to perform for each selected goal within one or more of the wellness dimensions.

22. The method of claim 21, further comprising:
allowing the user to set specific timeframes for achieving each selected goal within each wellness dimension; and
dynamically adjusting the recommended activities based on the selected timeframes.

23. The method of claim 17, further comprising generating notifications related to the personalized wellness journey of the new user, with the notifications comprising at least one of reminders, motivational messages, and progress updates.

24. The method of claim 17, wherein the generated suggestion for the new user based on the identified convergence further includes recommendations for one or more wellness related activities or events.

25. The method of claim 17, wherein identifying the convergence is based on at least one of user-defined wellness objectives, historical progress data, user preferences for interaction, and external wellness-related events or opportunities.

26. The method of claim 25, wherein the user preferences for interaction includes virtual meetings, shared wellness activities, mentoring or peer support relationships, collaborative learning sessions, and community event participation.

27. The method of claim 17, further comprising prioritizing the convergence based on a degree of complementarity between the at least one goal of the new user and the goals of the users in the database, with higher priority given to convergences that are likely to result in mutually beneficial outcomes.

28. The method of claim 17, further comprising tracking effectiveness of interactions facilitated by the convergence, and to use this data to refine future recommendations and improve accuracy of convergence identification.

29. The method of claim 17, further comprising providing a user-controlled option to manually search for potential convergences based on specific criteria, such as location, wellness focus, or desired interaction type.

30. A non-transitory computer readable medium having computer-executable instructions for causing a wellness management platform to perform steps comprising:
storing in a database wellness data on a plurality of users enrolled in a wellness management platform, with each user pursuing a personalized wellness journey generated by the wellness management platform to achieve at least one wellness goal, with the at least one wellness goal for each user having a progress status associated therewith that is being tracked and updated by the wellness management platform based on accomplishments of each user;
enrolling a new user in the wellness management platform, wherein the new user selects at least one wellness goal from a plurality of wellness dimensions;
generating a personalized wellness journey for the new user, with the personalized wellness journey providing recommendations to achieve the selected at least one wellness goal;
tracking progress of the new user on their personalized wellness journey based on accomplishments of the new user;
comparing the tracked progress of the new user on their personalized wellness journey to the wellness data stored in the database to identify a convergence with at least one of the users participating in the wellness management platform, wherein the identified convergence corresponds to overlapping or complementary wellness goals between the new user and the at least one user associated with the convergence;
generating a suggestion for the new user based on the identified convergence, wherein the suggestion includes at least one recommendation for joint activities, shared resources or communications with the at least one user associated with the identified convergence;
communicating the generated suggestion to the new user over a communications channel; and
providing user options via the generated suggestion to allow the new user to accept, modify, or decline the generated suggestion.
